(19) United States
(12) Patent Application Publication
(10) Pub. No.: US 2012/0070428 A1
(43) Pub. Date: Mar. 22, 2012

(54) TREATMENT OF VASCULARIZED PIGMENT EPITHELIAL DETACHMENT WITH ANTI-VEGF THERAPY

(75) Inventors: Clement K. Chan, Palm Desert, CA (US); Prema Abraham, Rapid City, SD (US)

(73) Assignee: Genentech, Inc., South San Francisco, CA (US)

(21) Appl. No.: 13/175,454

(22) Filed: Jul. 1, 2011

Related U.S. Application Data
(60) Provisional application No. 61/361,295, filed on Jul. 2, 2010.

(51) Int. Cl. A61K 39/395; A61P 27/02

(52) U.S. Cl. 424/133.1

(57) ABSTRACT

Methods for treating vascularized pigment epithelial detachment using anti-VEGF agents are disclosed.

### RELATED APPLICATIONS

This application claims benefit of priority under 35 U.S.C. §119(e) of U.S. Provisional Application No. 61/361,295, filed Jul. 2, 2010, the contents of which are incorporated herein by reference.

### FIELD OF THE INVENTION

This invention relates generally to treatment of human disease. More specifically, the invention relates to treatment of vascularized pigment epithelial detachment (vPED) associated with age-related macular degeneration (AMD).

### BACKGROUND OF THE INVENTION

Retinal pigment epithelial detachment (PED) is found in 10-12% of eyes diagnosed with AMD (Pauleikoff et al., Graefes Arch. Clin. Exp. Ophthalmol. 240: 533-38 (2002); Chan et al., Retina 27: 541-51 (2007)). The vascularized form of PED (vPED) consists of 70% of the PED (Pauleikoff et al., supra) and the visual prognosis associated with the natural history of a vPED is generally unfavorable.

Recently, there have been significant advances in the treatment of AMD, with several therapies having been recently approved—photodynamic therapy using verteporfin (Visudyne®); a VEGF-binding aptamer, pegaptanib (Macugen®); and an anti-VEGF antibody fragment, ranibizumab (Lucentis®). Based on these advances, a large multicenter randomized clinical trail (HARBOR study by Genentech, Inc., South San Francisco, Calif.) is being conducted to assess the efficacy of high-dose ranibizumab in treatment of individuals with AMD. However, previous studies with anti-VEGF agents has shown suboptimal and inconsistent anatomical and visual outcome when used for vPED. Although they may reduce the submacular hemorrhage and fluid associated with vPED, the vPED height decreases slowly and only rarely resolves completely (Chuang & Bird, Am. J. Ophthalmol. 105: 285-90 (1988); Pauleikoff et al., supra; Chan et al., supra, The Moorfields Macular Study Group, Br. J. Ophthalmol. 66: 1-16 (1982); Chen et al., Retina 27: 445-50 (2007)). Thus, there remains a significant unmet need in the treatment of vPED.

### SUMMARY OF THE INVENTION

The present invention is based in part on the surprising discovery that a vPED is rapidly flattened using a single dose of an anti-VEGF therapeutic where the dose administered is greater than the dose conventionally used for the treatment of AMD.

In one aspect, the invention provides a method of treating vascularized retinal pigment epithelial detachment (vPED) in a patient, comprising administering to the patient a high dose of an anti-VEGF therapeutic. In another aspect, the invention provides a method of flattening a vPED in a patient, comprising administering to the patient a high dose of an anti-VEGF therapeutic. In some embodiments, the anti-VEGF therapeutic is an anti-VEGF antibody, e.g.

ranibizumab. In some embodiments, the high dose is at least 2.0 mg, e.g. 2.0 mg. In some embodiments, the method further comprises administering a second dose of the anti-VEGF therapeutic. In some embodiments, the second doses is also a high dose, e.g. at least 2.0 mg, e.g. 2.0 mg.

### DETAILED DESCRIPTION OF THE PREFERRED EMBODIMENTS

The practice of the present invention will employ, unless otherwise indicated, conventional techniques of medical practice. Unless defined otherwise, technical and scientific terms used herein have the same meaning as commonly understood by one of ordinary skill in the art to which this invention belongs.

All references cited herein, including patent applications and publications, are incorporated by reference in their entirety.

Definitions

Before describing the present invention in detail, it is to be understood that this invention is not limited to particular compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “comprising” is intended to mean that the compositions and methods include the recited elements, but do not exclude others.

The terms “VEGF” and “VEGF-A” are used interchangeably to refer to the 165-amino acid vascular endothelial cell growth factor and/or related 121-, 189-, and 206- amino acid vascular endothelial cell growth factors, as described by Leung et al., Science 246: 1306 (1989), and Houck et al., Mol. Endocrin. 5: 1806 (1991), together with the naturally occurring allelic and processed forms thereof.

An “anti-VEGF therapeutic” or “anti-VEGF agent” refers to a molecule that inhibits VEGF-mediated angiogenesis, vasculogenesis, or undesirable vascular permeability. For example, an anti-VEGF therapeutic may be an antibody to or other antagonist of VEGF.

An “anti-VEGF antibody” is an antibody that binds to VEGF with sufficient affinity and specificity to be useful in a method of the invention. Preferably, the anti-VEGF antibody of the invention can be used as a therapeutic agent in targeting and interfering with diseases or conditions wherein the VEGF activity is involved. An anti-VEGF antibody will usually not bind to other VEGF homologues such as VEGF-B or VEGF-C, or other growth factors such as P1GF, PDGF or bFGF. A preferred anti-VEGF antibody is a monoclonal antibody that binds to the same epitope as the monoclonal anti-VEGF antibody A4.6.1 produced by hybridoma ATCC® HB 10709 and is a high-affinity anti-VEGF antibody. A “high-affinity anti-VEGF antibody” has at least 10-fold better affinity for VEGF than the monoclonal anti-VEGF antibody A4.6.1. Preferably the anti-VEGF antibody is a recombinant humanized anti-VEGF monoclonal antibody fragment generated according to WO 98/45331, including an antibody comprising the CDRs or the variable regions of Y0317. More preferably, anti-VEGF antibody is the antibody fragment known as ranibizumab (LUCENTIS ®). The anti-VEGF antibody ranibizumab is a humanized, affinity-matured anti-human

VEGF Fab fragment. Ranibizumab is produced by standard recombinant technology methods in E. coli expression vector and bacterial fermentation. Ranibizumab is not glycosylated and has a molecular mass of ˜48,000 daltons. See WO98/45331 and U.S. 2003/0190317.

The term “antibody” is used in the broadest sense and includes monoclonal antibodies (including full length or intact monoclonal antibodies), polyclonal antibodies, multivalent antibodies, multispecific antibodies (e.g., bispecific antibodies), and antibody fragments so long as they exhibit the desired biological activity.

“Treatment” refers to both therapeutic treatment and prophylactic or preventative measures. Those in need of treatment include those already with the disorder as well as those in which the disorder is to be prevented.

The term “effective amount” or “therapeutically effective amount” refers to an amount of a drug effective to treat a disease or disorder in a patient. In the case of vPED, the effective amount of the drug can reduce or prevent vision loss. For vPED therapy, efficacy in vivo can, for example, be measured by one or more of the following: assessing the mean change in the best corrected visual acuity (BCVA) from baseline to a desired time, assessing the NEI Visual Functioning Questionnaire, measuring the space observed beneath the retinal pigment epithelial detachment, e.g. by optical coherence tomography (OCT), measuring the size of the vPED surface area, e.g. by fundus photography (PD) and fluorescein angiography (FA), etc.

“Patient” for purposes of treatment refers to a human individual presenting with a disorder to be treated.

The term “high dose” refers to a dose of a therapeutic that is greater than that previously given. In the context of ranibizumab “high-dose” refers to a dose greater than 0.5 mg, e.g. about 1.0 mg or higher, about 2.0 mg or higher, etc.

The term “flattening a vPED” refers to reducing the space observed beneath the retinal pigment epithelial detachment until it is essentially normal and/or the area of the vPED is substantially reduced. In this context the space observed beneath the retinal pigment epithelial detachment in a vPED is “essentially normal” if either (a) a vPED which had a height ≧250 microns prior to treatment is reduced to less than 250 microns or (b) a vPED which had a height less than 250 microns prior to treatment is reduced by at least 50%. Similarly, the area of a vPED is “substantially reduced” in this context if the area is decreased by at least 50%. Preferably, both the height and the area of the vPED are reduced in the methods of the invention.

Modes of the Invention

It has been discovered that treatment of vPED using a high dose of a VEGF antagonist, including an anti-VEGF antibody (e.g., ranibizumab), results in rapid and sustainable flattening of the vPED.

Dosage and Administration

In methods of the invention, therapeutic effects of a VEGF antagonist are provided by administering the VEGF antagonist to a patient. In some embodiments, a single dose of a high dose VEGF antagonist is administered. In some embodiment, the first dose is followed by administration of additional dose(s) at regular intervals. For example, the dosages may be administered on a monthly basis in order to achieve the desired therapeutic effect and reduction in adverse effects. Alternatively, the additional dose(s) may be administered at the administering physician's discretion (e.g. pro re nata (PRN)). The specific time schedule can be readily determined by a physician having ordinary skill in administering the anti-VEGF therapeutic by routine adjustments of the dosing schedule within the method of the present invention.

Typically, the anti-VEGF therapeutic used in the methods of this invention is formulated by mixing it at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of antagonist, but preferably ranges anywhere from about 3 to about 8. Where the anti-VEGF therapeutic is an anti-VEGF antibody (e.g., ranibizumab), a suitable embodiment is a formulation at about pH 5.5.

The anti-VEGF therapeutic, e.g. an anti-VEGF antibody, for use herein is preferably sterile. Sterility can be readily accomplished by sterile filtration through (0.2 micron) membranes. Preferably, therapeutic peptides and proteins are stored as aqueous solutions, although lyophilized formulations for reconstitution are acceptable.

The anti-VEGF therapeutic may be formulated, dosed, and administered in a fashion consistent with good medical practice. Factors for consideration in this context include the particular disorder being treated, the particular patient being treated, the clinical condition of the individual patient, the cause of the disorder, the site of delivery of the agent, the method of administration, the time scheduling of administration, and other factors known to medical practitioners. The anti-VEGF therapeutic used in the methods of the invention is typically administered by intraocular, and/or intravitreal injection.

Efficacy of Treatment

The efficacy of the treatment of the invention can be measured by various endpoints commonly used in evaluating intraocular neovascular diseases. For example, vision loss can be assessed. Vision loss can be evaluated by, but not limited to, e.g., measuring by the mean change in best correction visual acuity (BCVA) from baseline to a desired time point (e.g., where the BCVA is based on Early Treatment Diabetic Retinopathy Study (ETDRS) visual acuity chart and assessment at a test distance of 4 meters), measuring the proportion of subjects who lose fewer than 15 letters in visual acuity at a desired time point compared to baseline, measuring the proportion of subjects who gain greater than or equal to 15 letters in visual acuity at a desired time point compared to baseline, measuring the proportion of subjects with a visual-acuity Snellen equivalent of 20/2000 or worse at a desired time point, measuring the NEI Visual Functioning Questionnaire, measuring the space observed beneath the retinal detachment, e.g. by OCT, etc. Ocular assessments can be done, e.g., which include, but are not limited to, e.g., performing eye exam, measuring intraocular pressure, assessing visual acuity, measuring slitlamp pressure, assessing intraocular inflammation, etc.

It is understood that the examples and embodiments described herein are for illustrative purposes only and that various modifications or changes in light thereof will be suggested to persons skilled in the art and are to be included within the spirit and purview of this application and scope of the appended claims.

### Example

After baseline examination, each study subject received 2.0 mg in 0.5 ml of ranibizumab (Genentech Inc, South San Francisco, Calif.) for treatment of a vPED in the right eye, and was followed for periodic evaluation, including standardized ETDRS visual acuity measurements, intraocular pressure measurements, slit-lamp biomicroscopy, indirect ophthalmoscopy, and Stratus optical coherence tomography (OCT) on a monthly basis, as well as fluorescein angiography (FA) and fundus photography (FP) every 3 months, according to the protocol of a Food and Drug Administration (FDA)-guided Investigator Sponsored Trial.

### Case Report

### Case 1

A 69 year-old Caucasian man presented with a 2-month history of moderate central vision deficit affecting the right eye. Ocular examination showed a best corrected visual acuity of right eye (RE): 20/50 and left eye (LE): 20/25. The anterior segment examination showed mild cortical and nuclear sclerotic cataracts. The posterior segment examination revealed mild macular drusen associated with mild nonexudative age-related macular degeneration in the left eye. However, there was a round subfoveal PED measuring one and a half disc areas in size, associated with mild submacular fluid, exudates and hemorrhage, consistent with a subfoveal vascularized pigment epithelial detachment (vPED). He received a single 2.0 mg intravitreal injection and within 10 days after the treatment, the subfoveal vPED was flattened, and the visual acuity of the right eye was 20/40. The eye received the same treatment on a monthly basis, and the PED remained flat after 6 months of follow-up.

### Case 2

An 86-year old Caucasian man reported a 6-month history of metamorphopsia affecting the left eye. During his retinal examination, his best corrected visual acuity was RE: 20/30, LE: 20/100. There were well-positioned posterior chamber implants in both eyes. There was also a posterior vitreous detachment in both eyes. Dilated fundus examination showed mild to moderate macular drusen and pigmentary changes in RE, and a large PED with a neovascular focus, consistent with a vPED in LE, confirmed by FP, FA, and OCT. His left eye was injected with a high (2.0 mg) dose of ranibizumab. Again, following a single intravitreal injection of the high-dose ranibizumab, a subsequent 4-week examination showed resolution of submacular fluid and flattening of the vPED. The vPED remained flattened at the 3-month follow-up examination.

The result from these two cases, along with three addition cases, are presented in greater detail below.

### Case 1:

Baseline vPED height: 158.73 microns
Baseline vPED Surface Area (SA): 10.82 mm²
Day-10 vPED height: 126.98 microns
(measured during non-protocol visit)
Week-4 vPED height: 95.24 microns
Week-8 vPED height: 63.4 microns
(>50% decrease from baseline)

### Week-12

vPED SA: 1.54 mm² (86% decrease in SA;
measured during non-protocol visit)
Comments: Clinical appearance of “flattening” of the vPED on exam, FP/FA, whereby a dramatic decrease in the bulk of the vPED has occurred, starting at Day-10. Further rapid decrease in the vPED continued on clinical examination at Week-4, and Week-8.

### Case 2:

Baseline vPED height: 380.95 microns
Baseline vPED SA: 10.01 mm²
Week-4 vPED height: 317.46 microns
Week-8 vPED height: 222.2 microns
Week-12 vPED height: 149.21 microns;
vPED SA: 5.64 mm² (44% decrease from baseline; measured during non-protocol visit)
Comments: The clinical appearance of dramatic decrease in height and SA of the vPED on exam, confirmed by FP and FA, was noted at Week-4. All features of the vPED continue to resolve rapidly after Week-4.

### Case 3:

Baseline vPED height: 146.03 microns
Baseline vPED SA: 2.55 mm²
Week-4 vPED height: 101.59 microns
Week-8 vPED height: Pending, but anticipate vPED height to be reduced to less than 50% of baseline, because a dramatic decrease in the bulk of the vPED is seen clinically and on FP/FA.
Week-12 vPED SA: 1.57 mm² (38% decrease from baseline)

### Case 4:

Baseline vPED height: 760.16 microns;
Baseline vPED SA: 4.7 mm²
Week-4 vPED height: 539.68 microns
Week-8 vPED height: 492.06 microns
Week-12 height: Pending
vPED SA: Pending
Comments: This case consists of a very large vPED with a substantial baseline height. Although the vPED heights at Week-4 and Week-8 are <50% decrease and >250 microns, the approximately 30% decrease in this “huge” vPED translates to obvious and dramatic improvement of the vPED on clinical appearance, FP and FA (despite the vPED height still >250 microns), indicating patient benefit even absent reaching these specific thresholds. Thresholds of 250 microns and 50% decrease will likely be attained by Week-12.

### Case 5:

Baseline vPED height: 634.92 microns;
Baseline vPED SA: 4.97 mm²
Week-4 vPED height: 228.57 microns
vPED SA: 4.08 mm² (decrease of 17.9%)
Week-8 vPED height: Pending
vPED SA: Pending
Comments: At Week-4, there are obvious and dramatic improvements in clinical appearance on the decrease in subretinal fluid, vPED height (below 250 microns), and vPED SA.

The favorable response of the 2.0 mg dose of ranibizumab for a vPED associated with this report is promising and demonstrates the utility of high dose ranibizumab for treatment of patients with vPED.

What is claimed is:

1. A method of treating vascularized retinal pigment epithelial detachment (vPED) in a patient, comprising administering to the patient a high dose of an anti-VEGF therapeutic.

2. The method of claim 1, wherein said anti-VEGF therapeutic is an anti-VEGF antibody.

3. The method of claim 2, wherein said anti-VEGF therapeutic is ranibizumab.

4. The method of claim 3, wherein said high dose is at least 2.0 mg.

5. The method of claim 4, wherein said high dose is 2.0 mg.

6. The method of any one of claims 1-5, further comprising administering a second dose of the anti-VEGF therapeutic.

7. A method of flattening a vPED in a patient, comprising administering to the patient a high dose of an anti-VEGF therapeutic.

8. The method of claim 7, wherein said anti-VEGF therapeutic is an anti-VEGF antibody.

9. The method of claim 8, wherein said anti-VEGF therapeutic is ranibizumab.

10. The method of claim 9, wherein said high dose is at least 2.0 mg.

11. The method of claim 10, wherein said high dose is 2.0 mg.

12. The method of any one of claims 7-11, further comprising administering a second dose of the anti-VEGF therapeutic.
